(19) United States
(12) Patent Application Publication
(10) Pub. No.: US 2026/0350795 A1
(43) Pub. Date: Nov. 5, 2026

(54) SYSTEMS AND METHODS FOR IMAGE-BASED CHARACTERIZATION OF TISSUE INTERNAL TO A PATIENT BODY

(71) Applicant: MOR RESEARCH APPLICATIONS LTD., Ramat Gan (IL)

(72) Inventors: Tom KONIKOFF, Ramat Gan (IL); Steven SHAMAH, Ramat Gan (IL)

(21) Appl. No.: 19/488,212

(22) PCT Filed: May 30, 2024
(86) PCT No.: PCT/IB2024/055282

§ 371 Date: Nov. 26, 2025

Related U.S. Application Data
(60) Provisional application No. 63/470,386, filed on Jun. 1, 2023.

(51) Int. Cl. G06T 7/00; G06T 7/10

(52) U.S. Cl. CPC G06T 7/0012; G06T 7/10; G06T 2207/20081; G06T 2207/30096

(57) ABSTRACT

Embodiments pertain to a computer program comprising program instructions for the execution of a method comprising the following steps, when the computer program is run on a computer: receiving image data descriptive of tissue internal to a mammalian; providing an image analysis engine with the received image data; and determining for the received image data, by the image analysis engine, whether imaged tissue includes a tissue anomaly or not. The steps may further comprise determining, for a detected anomaly, a type of anomaly. In some examples, the determining whether imaged tissue includes a tissue anomaly or not comprises determining a probability that imaged tissue comprises an anomaly.

### CROSS-REFERENCE TO RELATED APPLICATIONS

This application claims the benefit and/or priority from U.S. provisional application 63/470,386, filed Jun. 1, 2023, titled “COMPUTER AIDED DETECTION AND SEGMENTATION SYSTEM FOR ALL-TYPE ABNORMAL PANCREATIC LESIONS ON EUS”, and which is incorporated herein by reference in its entirety.

### BACKGROUND

Studies have demonstrated computer aided detection (CADe), to improve medical procedures, especially diagnostic procedures. In gastroenterology CADe has shown to significantly improve endoscopy. For example, increase adenoma detection rate (ADR) in colonoscopy, especially in novice examiners but overall, as well [11]. Therefore, incorporating AI in endoscopic ultrasound (EUS) is expected to be promising. Several, mostly pilot studies, have shown favorable results for preliminary AI systems in a variety of tasks such as anatomic structure recognition and differentiation between malignant and benign lesions [12-14].

The pancreas may harbor a wide variety of lesions many of which have significant clinical consequences. Some of these lesions necessitate early detection and characterization as they require prompt surgical therapy whereas others require long term surveillance [1, 2]. Pancreatic neoplasms are of specific concern as pancreatic ductal adenocarcinoma (PDAC) remains one of the most lethal cancers, projected to become the 2nd leading cause of cancer-related deaths in the US by the end of the decade [3-5]. Pancreatic cysts, specifically intraductal papillary mucinous neoplasms (IPMNs), are being increasingly detected in otherwise healthy patients and require careful surveillance due to their malignant potential[6]. Endoscopic ultrasound (EUS) is considered the most sensitive method of evaluating pancreatic lesions [7], enabling not only tissue sampling, but potentially also treating select lesions [8].

The description above is presented as a general overview of related art in this field and should not be construed as an admission that any of the information it contains constitutes prior art against the present patent application.

### BRIEF DESCRIPTION OF THE FIGURES

The figures illustrate generally, by way of example, but not by way of limitation, various embodiments discussed in the present document. For simplicity and clarity of illustration, elements shown in the figures have not necessarily been drawn to scale. For example, the dimensions of some of the elements may be exaggerated relative to other elements for clarity of presentation. Furthermore, reference numerals may be repeated among the figures to indicate corresponding or analogous elements. References to previously presented elements are implied without necessarily further citing the drawing or description in which they appear. The figures are listed below.

FIG. 1A shows a schematic block diagram illustration of a system configured to perform all-type tissue characterization in medical images, according to some embodiments.

FIG. 1B shows a flowchart of a method for performing all-type tissue characterization in medical images, according to some embodiments.

FIG. 2 is a schematic illustration of a segmentation evaluation metric, according to some embodiments.

FIG. 3A shows validation and training loss as a function of epochs, for training a transformer-based ML segmentation model, according to some embodiments.

FIG. 3B shows the Receiver Operating Characteristic (ROC) curve for the transformer-based model, according to some embodiments.

FIG. 3C shows training and validation loss as function of epochs, for training an CNN-based Model, according to some embodiments.

FIG. 3D shows the Receiver Operating Characteristic (ROC) curve for a trained CNN-based model, according to some embodiments.

FIG. 4 shows a segmentation output of the transformer-based model, according to some embodiments.

### DETAILED DESCRIPTION

The pancreas may harbor a wide variety of lesions many of which have significant clinical consequences. Some of these lesions necessitate early detection and characterization as they require prompt surgical therapy whereas others require long term surveillance [1, 2]. Pancreatic neoplasms are of specific concern as pancreatic ductal adenocarcinoma (PDAC) remains one of the most lethal cancers, projected to become the 2^{nd} leading cause of cancer-related deaths in the US by the end of the decade[3-5].

Pancreatic cysts, specifically intraductal papillary mucinous neoplasms (IPMNs), are being increasingly detected in otherwise healthy patients and require careful surveillance due to their malignant potential[6].

Endoscopic ultrasound (EUS) is considered the most sensitive method of evaluating pancreatic lesions[7], enabling not only tissue sampling, but also treating selected non-operable lesions [8]. A main drawback of EUS is that it requires specific advanced expertise and is significantly operator dependent, as evident by the inconsistent results in the various studies evaluating the accuracy of EUS for detection of pancreatic lesions[7, 9]. Unfortunately this is translated into an unacceptable high post-EUS pancreatic cancer rate [10]. In recent years, evidence showing artificial intelligence (AI) is capable of bridging operator dependency has accumulated. Studies have demonstrated computer aided detection (CADe) to increase ADR, especially in novice examiners but overall, as well [11]. Artificial intelligence can significantly mitigate this obstacle. Therefore, incorporating AI in EUS is expected to be promising. Several, mostly pilot studies, have shown favorable results for preliminary AI systems in a variety of tasks such as anatomic structure recognition and differentiation between malignant and benign lesions [12-14]. However, as EUS is comprised of essentially only 2D grayscale imaging, the task of detection on its own is not enough. Therefore, an ability to segment the findings is crucial to a successful EUS CADe system.

Endoscopic ultrasound (EUS) for example offers precise evaluation of pancreatic lesions but suffers from operator-dependency. Artificial intelligence (AI) has shown potential in increasing accuracy and bridging operator dependency such as in the case of adenoma detection rate in novice vs. experienced operators in colonoscopy.

Embodiments pertain to systems and methods for computer-aided (e.g., real-time, AI-based) detection, segmentation and, optionally, characterization, of a wide array of lesions in medical images during imaging of tissue and/or organs inside a patient body such as, for example, the pancreas. Imaging may, for example, be EUS-based, and/or be based any other medical imaging modality for imaging tissue internal to a patient body.

In some embodiments, system may include an analysis engine configured for analyzing medical images, e.g., in real-time, for detection and, optionally, segmentation and a wide array of (e.g., pancreatic) tissue anomalies (also: abnormalities) in the acquired medical images. Accordingly, in some examples, the system may be configured to provide an all-detection and all-lesion segmentation. Accordingly, the system may be configured to characterize identified and segmented image tissue into two or more lesions categories, classes and types. A lesion class may refer to a more specific division within a lesion category, and a type of lesion may refer to a more specific division of a class of lesion. For example, different lesion categories may pertain to normal pancreatic tissue vs. abnormal pancreatic tissue (i.e. cyst, solid lesions). A benign (a benign IPMN cyst) and malignant lesions (a malignant IPMN cyst) may be different classes of lesion

Further optionally, the analyzing may include characterizing a detected anomaly based on the acquired image(s) (also: “image-based” biopsy). Accordingly, in some examples, the analysis engine may be configured to characterize (e.g., classify) a detected anomaly, e.g., using “image-based” biopsy.

“Detection” may pertain to detecting, for at least one imaged scene in at least one corresponding field-of-view (FOV) of an image acquisition device, tissue that may be considered as “abnormal”, e.g., without drawing virtual boundaries for delineating the extent of the abnormal tissue. “Segmentation” may pertain to identifying and displaying boundaries relating to tissue identified as being abnormal for defining a virtual tissue segment of interest. Such virtual tissue segment of interest (or ROI) may be delineated by a virtual boundary or by margins, and displayed to the user in overlay or in combination with the imaged tissue region. The system may further be configured to perform “image-based biopsy”, which may include determining characteristics relating to the imaged tissue segment.

In some embodiments, the system may employ one or more trained ML models configured to perform detection, segmentation and, optionally, “image-based” biopsy of the segmented lesion (outputting both a direct diagnosis of “cyst”, a diagnosis of type of cyst, such as IPMN, and the classification of “benign” or “malignant” diagnosis). In some embodiments, the one or more ML-models may include one or more classifiers for identifying or detecting, based on an image dataset, tissue anomalies such as, for example, a lesion, distinguish between various types of lesions, and clinically characterize an identified lesion. In some examples, the ML-model may be employed in conjunction with a rule-based approach.

In some examples, the classifier may employ or include one or more ML models which were trained based on a training image dataset that was associated, e.g., in a supervised manner, by providing virtual boundaries relating to abnormal tissue shown in medical image datasets. In some examples, virtual tissue segments may be associated with labels relating to tissue segment characterization (e.g., “biopsy” information).

In some embodiments, detection of abnormal tissue may include distinguishing between normal and abnormal tissue by the system. In some embodiments, the system may be configured to distinguish between different types of abnormal tissue (e.g., benign or normal lesions, tissue inflammations, abnormal lesions). In some embodiments, the system may be configured to distinguish between different types of tissue anomalies (e.g., between malignant and non-malignant lesions). In some examples, an abnormal lesion may pertain to any abnormal pancreatic tissue be it benign (such as, for example, cust), or malignant (such as, for example, adenocarcinoma).

In some embodiments, the systems and methods may be configured to perform “image-based biopsy” which may include determining, for example by a trained machine learning model (ML), lesion-related characteristics. Determining tissue-related characteristics may include, for example, identifying a type of abnormal tissue, abnormal tissue classification, staging, distinguishing between anomalies related to potentially cancerous lesions vs anomalies related to non-cancerous anomalies.

Furthermore, in some embodiments, a probability may be determined in association with a lesion to transform from a non-cancerous (e.g., normal lesion) to a cancerous (e.g., malignant or non-malignant) lesion. In some embodiments, an onset of probability may be determined that certain tissue segment may transform from a non-malignant to a malignant lesion. In some embodiments, a probability may be determined for which within a future time interval a lesion transforms into a malignant lesion. In some embodiments, a future time interval may be determined for which a transformation of the lesion into a malignant lesion is within a certain probability.

In some embodiments, the systems and methods may output a probability-time tuple relating to characteristics of one or more lesions, or a probability-time-anatomical location tuple relating to lesion characteristics.

In some embodiments, detection of abnormal tissue (e.g., lesions, inflamed tissue), as well as segmentation and/or characterization thereof may be performed intra-operatively, and/or post-operatively.

In some embodiments, the system may be configured to guide a user of an endoscopy imaging device to improve detection of abnormal tissue, improve segmentation, and/or improve image-based biopsy.

In some embodiments, the systems and methods may include a decision support module configured to provide an output indicative of actionable suggestion or instructions relating to follow-up diagnostic and/or treatment procedures to be performed on a patient. Such instructions may relate, for example, relate to a time frame within which an additional imaging procedure is to be performed for monitoring development of an normal lesion, and abnormal lesion, a cancerous lesion, and/or the like; lesion treatment recommendations (e.g., surgical intervention, medication); regarding the performance of conducting additional lesion imaging using different imaging modalities (e.g., CT); and/or the like. In some embodiments, recommendations may be aimed at reducing false-positives detection and/or false-negative abnormal tissue detection and/or tissue characterization. For example, the system may output a probability or confidence level score with respect to the detection and/or characterization of tissue anomaly. For example, if for an image-based biopsy, a probability of tissue malignancy tissue is below a certain threshold value, the system may recommend to conduct additional diagnostic follow-up tests.

In some embodiments, the system may be configured to render a 3D representation of a segment.

In some embodiments, an ML model employed by a medical imaging analysis engine in conjunction with a first imaging modality may be trained based on medical imagery of the same imaging modality. For example, an ML model employed for analyzing EUS images by the analysis engine may be trained using supervised ML, for example, based on multiple EUS images. In some embodiments, the ML model may also be trained in an unsupervised manner.

In some embodiments, an ML model employed by a medical image analysis engine in conjunction with a first imaging modality may be trained based on medical imagery of a second imaging modality which is different from the first imaging modality. For example, the ML model employed during EUS imaging procedures may be additionally or alternatively be trained using an non-EUS image modalities, such as, for example, Computer-Tomography (CT) images.

In some embodiments, a training image dataset may be segmented for training the ML learning to perform ML-based image segmentation. In some examples, the segmentation of the images may be performed, for example, on endoscopically and/or non-endoscopically acquired image datasets such as, for example, EUS images; CT images; X-ray images, MRI images; and/or the like. In some examples, the training image datasets may be anonymized training image datasets.

In some examples, the training image datasets may be labelled. Such labels may pertain to additional medical, physiological and/or socio-economic and/or behavioral patient characteristics such as, for example, gender, age, race, height, BMI, smoking habits, drinking habits, medical history, and/or the like.

In some examples, the machine learning model may be adapted by evaluating virtual margins and/or labels produced by a test dataset. The validation measures may include, for example, accuracy, recall and/or precision, with respect to real labels and/or virtual margins on an image dataset of labeled data.

In some embodiments, the system may be configured to perform image dataset analysis using heuristics models. Further, in some instances, the machine learning and heuristics models may be combined into a hybrid model for analyzing the image dataset.

In some embodiments, datasets may be excluded as training datasets, based on one more exclusion criteria. In some examples, the system may be configured to automatically include and/or exclude training datasets provided for training a classifier. Exclusion criteria may include, for instance, extremely altered anatomy.

As used herein the term “machine learning” refers to a procedure embodied as a computer program configured to induce patterns, regularities, and/or rules from previously collected data to develop an appropriate response to future data or describe the data in some meaningful way.

Examples of machine learning procedures suitable for the present embodiments, include, without limitation, clustering, association rule algorithms, feature evaluation algorithms, subset selection algorithms, support vector machines, classification rules, cost-sensitive classifiers, vote algorithms, stacking algorithms, Bayesian networks, decision trees, neural networks, instance-based algorithms, linear modeling algorithms, k-nearest neighbors (KNN) analysis, ensemble learning algorithms, probabilistic models, graphical models, logistic regression methods (including multinomial logistic regression methods), gradient ascent methods, singular value decomposition methods and principle component analysis.

The machine learning procedure used according to some embodiments of the present invention is a trained machine learning procedure, which provides output that is related, e.g., non-linearly, to the parameters with which it is fed.

In some embodiments, a machine learning procedure can be trained according to some embodiments of the present invention by feeding a machine learning training program with parameters that characterizes image datasets descriptive of tissue internal to subjects. Once the data is fed, the machine learning training program generates a trained machine learning procedure or forms a part of a ML module. In some examples, the trained ML module can be used without the need to re-train it. In some other examples, the trained ML module may be further trained and tested, e.g., on-the-fly, while performing an diagnostic imaging procedure on a subject.

In some embodiments, in a training phase, various images relating to a certain region internal to a patient body of a same subject may be annotated by a plurality of annotators or experts (e.g., trained physicians of a same field) for deriving one or more consensus ROIs relating to the acquired images. With respect to an image dataset of a patient, a consensus ROI may be derived based on a plurality of ROIs which are defined by a plurality of different annotators. Such consensus ROI may be the overlapping region of the plurality of ROIs defined by the plurality of annotators in the training stage. In other words, a consensus ROI may be common to the ROIs defined by the plurality of annotators.

In some embodiments, at least one of the consensus ROI may be annotated (also: labelled) by each of the annotators. In some embodiments, only consensus ROIs annotated with same labels by the plurality of annotators may be used in the training stage. In some embodiments, labels provided by the experts in the training phase may be validated based on biopsy, PET CT, and/or other diagnostic methods. In some embodiments, ML-model outputs may be tested against biopsy, PET CT and/or other diagnostic methods.

In some examples, ground truth for segmentation training data may be, for example, an area of consent of the annotators, as discussed herein. In some examples, ground truth of lesion classification may be, for example, biopsy.

In some embodiments, a labelled consensus ROI may be used for training the ML-model for detection of features in, and segmentation of image datasets. The trained ML-model may then be employed by the image analysis engine of the system. The system may herein also be referred to as a Computer-Aided Detection (CADe) System.

In some embodiments, ML-models employed may include, for example, of the ML-model may include, for example, benchmarking one or more deep learning options for imaging segmentation tasks such as, for example, convolutional neural network (CNN), and a visual transformer (ViT). Convolutional neural networks apply convolutional filters to input data, which helps in detecting patterns, edges, and textures, thus enabling the model to learn hierarchical feature representations useful for tasks like recognition.

In some embodiments, UperNet ConvNeXt may be employed for semantic segmentation. UperNet ConvNeXt combines UperNet framework for semantic segmentation that leverages a ConvNeXt backbone.

In some embodiments, visual transformers may be employed. Visual transformers are a type of neural network architecture that applies the principles of self-attention, originally used in natural language processing, to visual tasks. They work by dividing an image into patches and using attention mechanisms to dynamically focus on different parts of the image, enabling the model to capture global dependencies and understand complex visual relationships. As a Visual transform, SegFormer may be employed for semantic segmentation.

In some embodiments, the models employed for training the ML model may be pretrained. In some examples, the hyperparameter tuning, including optimization algorithm, learning rate, and training epoch count (which represents the number of times the entire training set is passed through the model during training) may be performed as accepted, by programmatic search across a range of values. The training parameters used in the training phase may include, for example: (1) number of epochs (including early stopping on IoU metric), (2) batch size (which determines the number of images used in each training iteration), (3) learning rate. In some examples, cross entropy (pixel-wise) may be employed as the loss function.

In some embodiments, the training set may contain 70% of the dataset, while the validation set may contain 15% and the remaining 15% may be allocated to the holdout test set. In some examples, data splitting may be performed using stratified sampling.

In some embodiments, mode performance may be measured on the test set and reported using methods including sensitivity, specificity, and accuracy, e.g., based on the intersection over union (IoU). The IoU is an evaluation metric that is calculated from the area of overlap (of the predicted and labeled findings) divided by area of union (of the predicted and labeled findings), and may be reported per pixel. Model performance may further be evaluated on the test set using, for example, measures of area under the operator characteristic (AUROC) curve, positive predictive value (PPV), negative predictive value (NPV), overall accuracy, F1 score, and/or the like.

In some embodiments, a CADe system for pancreatic EUS may have an AUROC of 0.95 (CI 95 0.92-0.98) and mean IoU of 0.73 (0.94, CI 95 0.91-0.97, for normal tissue and 0.51, CI 95 0.44-0.58, for findings) per pixel.

The CADe system, especially given a high IoU for abnormal findings (e.g., 0.94, CI 95 0.91-0.97), may increase detection, and segmentation of all abnormalities (e.g., cysts, NETs, and Malignancies) within the pancreatic parenchyma and thus aid in the reduction of the pancreatic cancer miss rate. In some embodiments, the performance of the CADe system may surpass those of fine needle biopsy (FNB). In FNB, there may a high rate of insufficient material for histologic diagnosis (6.6%), sometimes necessitating re-biopsy (7.3%), and higher rate of false negative (14.4%) most of them with PDAC [11]. Therefore, the CADe system, e.g., for EUS can potentially lower the lesion miss rate and better focus the FNB procedure, thus reducing the need for repeat biopsy and associated adverse events.

The CADe system, according to some embodiments, may facilitate and/or improve the detection of lesions that may otherwise be missed, improve tissue acquisition, and decrease the need for repeat procedures and associated risks of adverse events. This may ultimately improve the early detection of pancreatic malignancies, especially in high-risk populations and in low-volume centers.

In some embodiments, the system includes at least one processor; and
at least one memory configured to store data and software code portions executable by the at least one processor to cause to perform the following:
receiving data that are descriptive of at least one image of a mammalian's tissue internal to the mammalian's body,
providing a trained ML module with the received data; and
determining for the data, by the trained ML module, whether the imaged tissue contains an abnormal tissue or not.

In some embodiments, the system may be further be configured to segment the tissue to indicate boundaries relating to the detected abnormal tissue.

In some embodiments, the system may be configured to output, for tissue segment, a type of lesion.

In some embodiments, the system may be configured to output, for detected abnormal tissue, a type of lesion.

In some embodiments, the system may be configured to output, for detected abnormal tissue, the boundaries thereof.

In some embodiments, the system may be configured to distinguish between different tissue anomalies.

Optionally, the mammalian is a human subject.

In some embodiments, the system may provide, based on the performed analysis, with one or more intervention and/or diagnostic procedure recommendations relating to a detected tissue anomaly.

In some embodiments, the system may produce at least one first output indicative of a probability that a non-cancerous tissue anomaly is expected to develop into a cancerous lesion.

In some embodiments, the terms “lesion” and “tissue anomaly” may herein be used interchangeably.

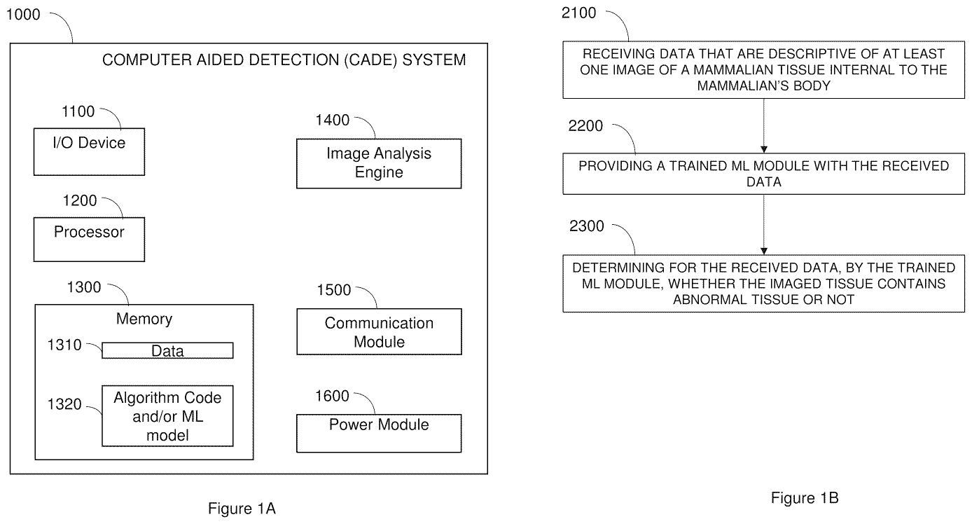

Referring now to FIG. 1A, a CADe system 1000 may comprise an I/O device 1100, a processor 1200 and a memory 1300.

In some example implementations, CADe system 1000 may provide a user thereof with outputs via I/O device 1100 comprising one or more output devices. The one or more output devices may include, for example, devices that are configured to convert electrical signals into outputs that can be sensed as output by a human, such as sound, light, and/or touch. Output devices can include display screens, and/or audio output device(s) such as, for example, speaker(s) and/or earphones.

I/O device 1100 may further include one or more input devices which are configured to receive any type of data and/or information by converting, for example, or machine-generated signals and/or human-generated signals such as physical movement, physical touch and/or pressure, and/or the like, into electrical signals as input data into the computing system. Examples of such input devices include touch screens, microphones, hand gesture tracking devices, hand-held pointing devices (e.g., computer mouse, stylus), hand-held imaging devices (e.g., endoscopes such as, for example, endoscopic ultrasound imaging devices), and/or the like.

I/O device 1100 may be employed to access data and/or information generated by the system 1000 and/or to provide inputs including, for instance, control commands, operating parameters, queries and/or the like. For example, I/O device 1100 may allow a user of a system to receive or medical images of a patient and/or other patient-related information.

System 1000 may further include a processor 1200 and a memory 1300 which is configured to store data 1310 (e.g., patient data) and algorithm code and/or a machine learning (ML) model 1320. Processor 1200 may be configured to execute algorithm code and/or apply machine learning (ML) model 1320 for the processing of data 1310 resulting in the implementation of an CADe or image analysis engine 1400. Analysis engine 1400 may be configured to provide an output (e.g., an image) indicative of tissue lesion, location and, optionally, margins thereof. In some examples, the output may include information characterizing the type of lesion.

The term “processor”, as used herein, may additionally or alternatively refer to a controller. Processor 1200 may be implemented by various types of processor devices and/or processor architectures including, for example, embedded processors, communication processors, graphics processing unit (GPU)-accelerated computing, soft-core processors, quantum-based processor and/or general-purpose processors.

Memory 1300 may be implemented by various types of memories, including transactional memory and/or long-term storage memory facilities and may function as file storage, document storage, program storage, or as a working memory. The latter may for example be in the form of a static random-access memory (SRAM), dynamic random-access memory (DRAM), read-only memory (ROM), cache and/or flash memory. As working memory, memory 1300 may, for example, include, e.g., temporally based and/or non-temporally based instructions. As long-term memory, memory 1300 may for example include a volatile or non-volatile computer storage medium, a hard disk drive, a solid-state drive, a magnetic storage medium, a flash memory and/or other storage facility. A hardware memory facility may for example store a fixed information set (e.g., software code) including, but not limited to, a file, program, application, source code, object code, data, and/or the like.

System 1000 may further comprise at least one communication module 1500 configured to enable wired and/or wireless communication between the various components and/or modules of the apparatus and which may communicate with each other over one or more communication buses (not shown), signal lines (not shown) and/or a network infrastructure. Communication module 1500 may be configured for enabling communication using one or more communication formats, protocols and/or technologies such as, for example, to internet communication, optical or RF communication, telephony-based communication technologies and/or the like. In some examples, communication module 1500 may include I/O device drivers (not shown) and network interface drivers (not shown) for enabling the transmission and/or reception of data over a network. A device driver may, for example, interface with a keypad or to a USB port. A network interface driver may for example execute protocols for the Internet, or an Intranet, Wide Area Network (WAN), Local Area Network (LAN) employing, e.g., Wireless Local Area Network (WLAN)), Metropolitan Area Network (MAN), Personal Area Network (PAN), extranet, 2G, 3G, 3.5G, 4G, 5G, 6G mobile networks, 3GPP, LTE, LTE advanced, Bluetooth© (e.g., Bluetooth smart), ZigBee™, near-field communication (NFC) and/or any other current or future communication network, standard, and/or system.

System 1000 may further include a power module 1600 for powering the various components and/or modules and/or subsystems of the apparatus. Power module 1600 may comprise an internal power supply (e.g., a rechargeable battery) and/or an interface for allowing connection to an external power supply.

It will be appreciated that separate hardware components such as processors and/or memories may be allocated to each component and/or module of system 1000. However, for simplicity and without be construed in a limiting manner, the description and claims may refer to a single module and/or component. For example, although processor 1200 may be implemented by several processors, the following description will refer to processor 1200 as the component that conducts all the necessary processing functions of system 1000.

Functionalities of system 1000 may be implemented fully or partially by a multifunction mobile communication device also known as “smartphone”, a mobile or portable device, a non-mobile or non-portable device, a digital video camera, a personal computer, a laptop computer, a tablet computer, a server (which may relate to one or more servers or storage systems and/or services associated with a business or corporate entity, including for example, a file hosting service, cloud storage service, online file storage provider, peer-to-peer file storage or hosting service and/or a cyberlocker), personal digital assistant, a workstation, a wearable device, a handheld computer, a notebook computer, a vehicular device, a non-vehicular device and/or a stationary device. For example, some of the functionalities of analysis engine 1400 may be implemented on-premises (e.g., in a hospital or other clinical facility), and some by devices, apparatuses and/or system which are located off-premises (e.g., the “cloud”). Alternative configurations may also be conceived.

Additional reference is now made to FIG. 1B. As indicated by block 2100, a method for performing image-based characterization of tissue internal to a patient body may include, for example, receiving image data that are descriptive of at least one image of a mammalian's tissue internal to the mammalian's body.

As indicated by block 2200, the method may further include, for example, providing a trained ML module with the received image data.

As indicated by block 2300, the method may include, for example, determining for the received image data, by the trained ML module, whether the imaged tissue contains abnormal tissue or not. In some embodiments, the method may include distinguishing between different types of abnormal tissues (also: tissue anomalies).

In some examples, the method may further include segmentation, by the ML model, the received image data.

In some examples, the ML model employed may be trained based on a plurality of consensus ROIs of image data annotated and, optionally, labelled by a plurality of different experts in the same field, for a respective plurality of subjects and corresponding organ tissue, e.g., intraoperatively and/or postoperatively. For example, a plurality of consensus ROIs may be identified by at least two experts for a plurality of pancreases of a respective plurality of subjects. For a selected tissue and/or organ (e.g., pancreas) of multiple subjects, the ML-model(s) may be trained based on a plurality of consensus ROIs derived based on ROIs individually delineated by at least two different experts, for performing segmentation. A consensus ROI may represent an overlapping region of at least two different ROIs annotated by the at least two different experts.

In some examples, a consensus or overlapping training ROI may be obtained based on two or more ROIs annotated by two or more experts. In some embodiments, training ROIs for training the ML-model may be based on consensus ROIs and/or based on individual ROIs that do not take into account a consensus ROI. In some examples, training data of unity ROIs and of consensus ROIs may be collectively employed for training an ML model. In some embodiments, at least two of a plurality of consensus ROIs may be derived by at least two different groups of annotators. For example, a first consensus ROI may be derived based on individual ROIs provided by a first group of annotators, and a second consensus ROI may be derived based on individual ROIs provided by a second group of annotators. In some examples, the first group of annotators and the second group of annotators may have at least one non-common member. In some examples, all members of the first group of annotators may be different from the members of the second group of annotators. In some examples, at least one member of the first group of annotators may be different from the members of the second group of annotators.

By relying on a consensus or overlapping ROI for training an ML-model to perform tissue segmentation the probability of a false-positive identification of an anomalous tissue segments and/or a false-negative identification of anomalous tissue segments may be reduced, compared to probabilities of false-positive and false-negative identifications if no consensus region was taken into consideration.

It is noted that although embodiments and examples discussed herein may pertain to the pancreas, this should by no means be construed in a limiting manner. Accordingly, the systems and methods disclosed herein for training an ML model, and for the detection, segmentation and image-based biopsy of tissue may analogously be applicable to other organs such as, for example, the liver, kidneys, bladder, prostate, bones, skeleton, lung, and/or the like. A label may include a “normal lesion”, an “abnormal lesion”, a type of abnormal lesion such as, for example, “cancerous lesion” (e.g., cyst), “non-cancerous lesion”, etc. Type may further include, for example, specific diagnosis such as IPMN, mucinous cyst, neuroendocrine tumor, or adenocarcinoma, etc. Optical biopsy may pertain to identifying a specific type of lesion, e.g., “malignant lesion”, “non-malignant lesion”, and/or the type of malignant or non-malignant lesion such as, for example, serous cyst, IPMN (both benign or malignant), neuroendocrine tumor, pseudocyst, adenocarcinoma, metastasis, etc.

### Examples

Reference is now made to the following examples, which together with the above descriptions illustrate some embodiments in a non-limiting fashion.

### Methods:

### Study Design and Patients:

This single-center study included a prospectively-collected, retrospectively-analyzed data set of EUS images. We included adult (>18 years old) patients who underwent EUS of the pancreas with or without tissue acquisition at the Rabin Medical Center (RMC) between January 2020 and July 2021. Procedures were performed by five expert endoscopists (using the PENTAX EG38-J10UT therapeutic linear ultrasound video endoscope). Patients were followed through January 2022 to exclude diagnostic errors. The study was approved by the institutional ethics committee (No. 0949-20-RMC). Due to the study's retrospective nature informed consent was wavered. All authors accessed the study data, reviewed, and approved the final manuscript.

### Data Preparation and Pre-Processing:

For the purpose of real-world setting all images per case were retrieved and none were excluded from the algorithm training process unless there was a technical error. Findings were annotated (delineated) by two expert endoscopists (>5 years of EUS with at least >250 EUS procedures a year) using the SuperAnnotate platform (SuperAnnotate.com). The software enables scrolling through different contrast options and extreme zoom to detect subtle details in the echo-texture. The annotators had access to the procedure report, histology, and electronic medical record to further increase accuracy of the annotation process. The resulting annotated images (resolution 448×576 in grayscale) were then processed to yield the ‘consensus region’ mask used for model development.

### Model Development:

Although our system both detects and segments findings, for the purpose of keeping in terms with current nomenclature in the field of AI in endoscopy, CADe was the term used to describe this EUS AI in our study. We benchmarked the two leading popular deep learning options for imaging segmentation tasks; convolutional neural network (CNN) and visual transformer. Convolutional neural networks apply convolutional filters to input data, which helps in detecting patterns, edges, and textures, thus enabling the model to learn hierarchical feature representations useful for tasks like recognition. We used UperNet ConvNeXt, which combines UperNet framework for semantic segmentation that leverages a ConvNeXt backbone. Visual transformers are a type of neural network architecture that applies the principles of self-attention, originally used in natural language processing, to visual tasks. They work by dividing an image into patches and using attention mechanisms to dynamically focus on different parts of the image, enabling the model to capture global dependencies and understand complex visual relationships. We used SegFormer, which is specifically tailored for semantic segmentation. Both models were pretrained. The hyperparameter tuning, including optimization algorithm, learning rate, and training epoch count (which represents the number of times the entire training set is passed through the model during training) was performed as accepted, by programmatic search across a range of values. The training parameters used in the training phase were: (1) number of epochs (including early stopping on IoU metric), (2) batch size (which determines the number of images used in each training iteration), (3) learning rate. The loss function used is the cross entropy (pixel-wise). The models were developed in Python using Cuda and the NVIDIA GeForce RTX 3090 GPU. The training set contained 70% of the dataset, while the validation set contained 15% and the remaining 15% was allocated to the holdout test set. Data splitting was done using stratified sampling.

### Model Evaluation:

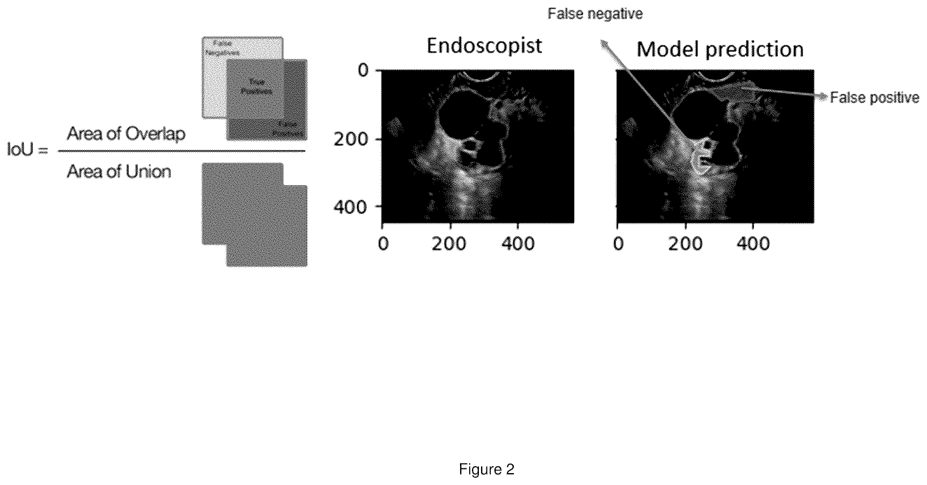

Model performance was measured on the test set and reported using accepted methods including sensitivity, specificity, and accuracy based on the intersection over union (IoU). The IoU is considered a rigorous evaluation metric that is calculated from the area of overlap (also: consensus ROI) (of the predicted and labeled findings) divided by area of union (of the predicted and labeled findings) as described in FIG. 2 and is reported here per pixel. We further evaluated the model performance on the test set using the accepted measures of area under the operator characteristic (AUROC) curve, positive predictive value (PPV), negative predictive value (NPV), overall accuracy, and F1 score.

### Results:

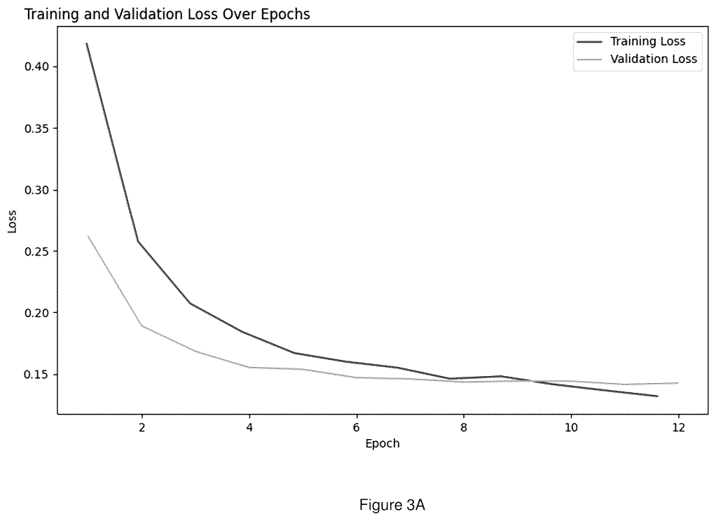

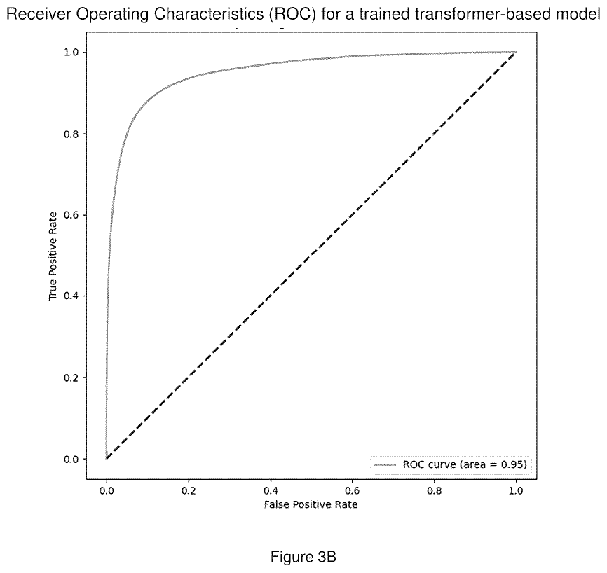

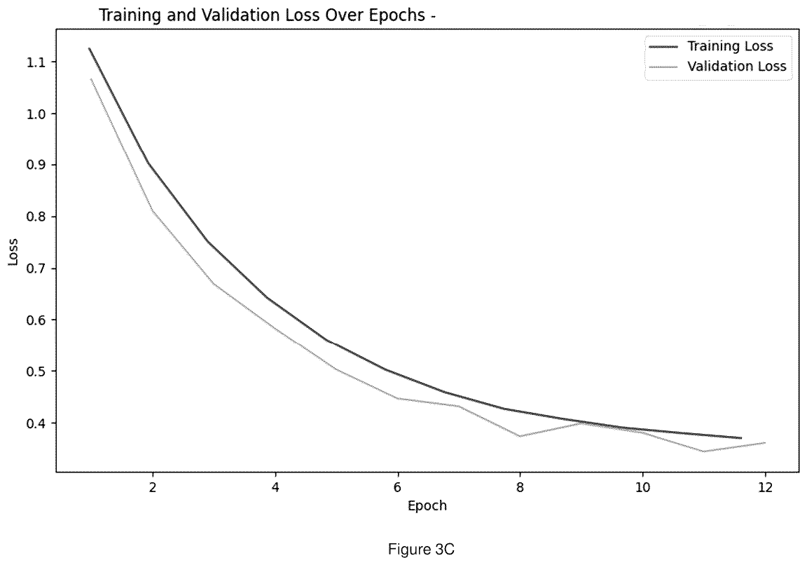

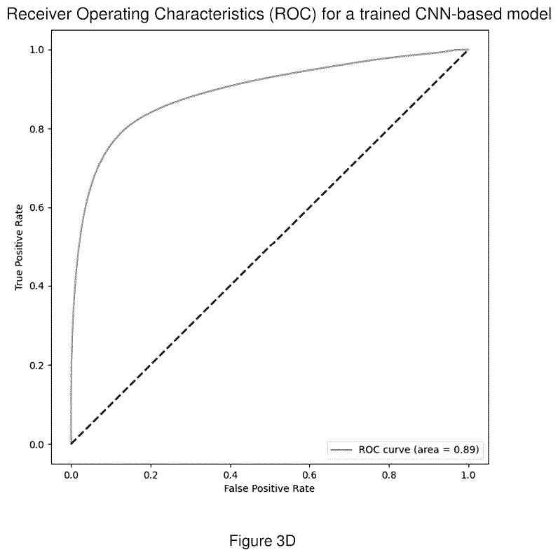

A total of 182 cases were included in the study. Of those, 17 were excluded due to inconclusive histology or missing data. A final 165 cases were used for model development and evaluation. Diagnoses included 68 malignancies (62 pancreatic adenocarcinomas, one IPMN with high grade dysplasia [HGD], one PanIN grade 3, two lymphomas, one adenoma with HGD, one metastasis), 19 neuroendocrine tumors (grades 1-3), 48 benign cysts (including IPMNs, serous cysts, mucinous cysts), four cases of chronic or acute pancreatitis, seven cases of fatty normal pancreas (with no malignancy or other diseases on follow up), and 33 benign lesions (including walled off necrosis, fibrosis, adjacent lymphadenopathy or undetermined findings that on follow up were ruled out for malignancy). For purpose of stratification, the cases were grouped into Cysts (serous, mucinous, and IPMNs), Solids (adenocarcinoma, NETs, lymphoma, and metastases), Inflammation, Mixed (containing more than one group) and ‘Other’ (PanINs, benign lesions including undetermined benign lesions). The total number of images were 1,497 (1,034, 260, and 203 in the training, validation and test sets, respectively). The number of trainable parameters for the UperNet ConvNeXt were 81,763,236. The number of trainable parameters for the SegFormer were 3,714,658 (^{~}3.4M for the encoder and ^{~}0.4M for the decoder). The UperNet ConvNeXt model was able to detect and segment abnormal pancreatic tissue with an overall accuracy of 0.93 (CI 95 0.90-0.97) and a AUROC of 0.89 (CI 95 0.85-0.93). The mean IoU was 0.66 (0.93, CI 95 0.90-0.96, for normal tissue and 0.40, CI 95 0.34-0.47, for findings) which corresponds to a PPV of 0.72 (Cl 95 0.66-0.78), an NPV of 0.95 (CI 95 0.92-0.98), a sensitivity of 0.48 (Cl 95 0.41-0.55), a specificity of 0.98 (CI 95 0.96-1) and an f1 score of 0.58 (Cl 95 0.51-0.64) per pixel. The SegFormer model was able to detect and segment abnormal pancreatic tissue with an overall accuracy of 0.95 (CI 95 0.92-0.98) and a AUROC of 0.95 (Cl 95 0.92-0.98). The mean IoU was 0.73 (0.94, CI 95 0.91-0.97, for normal tissue and 0.51, CI 95 0.44-0.58, for findings) which corresponds to a PPV of 0.82 (CI 95 0.76-0.87), an NPV of 0.96 (CI 95 0.93-0.98), a sensitivity of 0.57 (CI 95 0.51-0.64), a specificity of 0.98 (CI 95 0.97-1.0) and an f1 score of 0.67 (CI 95 0.61-0.74) per pixel. The loss and AUROC graphs of the models are shown in FIGS. 3a-d and the models metrics are shown in Table 1 below:

TABLE 1
Model's performance on the test set

| | | | Mean | | | | | |
|---|---|---|---|---|---|---|---|---|
| Model | Accuracy | AUROC | IoU | Sensitivity | Specificity | NPV | PPV | F1 score |
| SegFormer | 0.95 | 0.95 | 0.73 | 0.57 | 0.98 | 0.96 | 0.82 | 0.67 |
| | (0.92-0.98) | (0.92-0.98) | (0.68-0.78) | (0.51-0.64) | (0.97-1.0) | (0.93-0.98) | (0.76-0.87) | (0.61-0.74) |
| UperNet | 0.93 | 0.89 | 0.66 | 0.48 | 0.98 | 0.95 | 0.72 | 0.58 |
| ConvNeXt | (0.90-0.97) | (0.85-0.93) | (0.62-0.71) | (0.41-0.55) | (0.96-1) | (0.92-0.98) | (0.66-0.78) | (0.51-0.64) |

IoU: intersection over union. All CI are given in 95%.

FIG. 3A shows validation and training loss as a function of epochs, for training a transformer-based ML segmentation model, according to some embodiments.

FIG. 3B shows the Receiver Operating Characteristic (ROC) curve for a trained transformer-based model, according to some embodiments.

FIG. 3C shows training and validation loss as function of epochs, for training an CNN-based Model, according to some embodiments.

FIG. 3D shows the Receiver Operating Characteristic (ROC) curve for a trained CNN-based model, according to some embodiments.

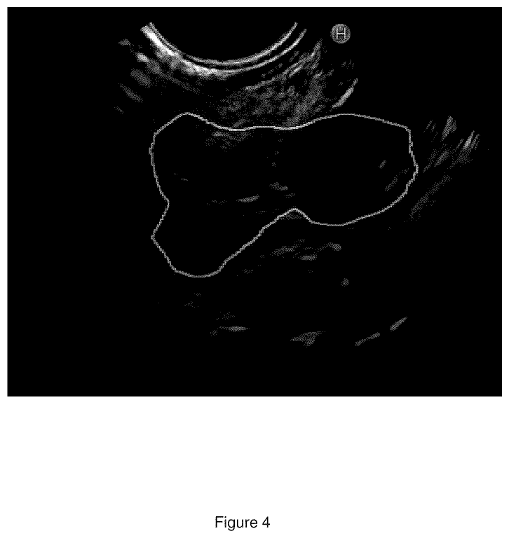

FIG. 4 shows a segmentation output of the transformer-based model, according to some embodiments. In the example shown, the output delineates an 11-mm neuroendocrine tumor (NET)—taken from a real-time video.

### ADDITIONAL EXAMPLES

Example 1 pertains to computer program comprising program instructions for the execution of a method comprising the following steps, when the computer program is run on a computer:
receiving image data descriptive of tissue internal to a mammalian;
providing an image analysis engine with the received image data; and
determining for the received image data, by the image analysis engine, whether imaged tissue includes a tissue anomaly or not.

Example 2 includes the subject matter of example 1 and, optionally, wherein the steps comprise: determining, for a detected anomaly, a type of anomaly.

Example 3 includes the subject matter of any one or more of the examples 1 to 2, and optionally, wherein the determining whether imaged tissue includes a tissue anomaly or not comprises determining a probability that imaged tissue comprises an anomaly.

Example 4 includes the subject matter of any one or more of the examples 1 to 3 and, optionally, wherein the determining whether imaged tissue includes a tissue anomaly or not comprises determining a probability that imaged tissue does not comprise an anomaly.

Example 5 includes the subject matter of any one or more of the examples 1 to 4 and, optionally, wherein the steps further comprise: for a detected tissue anomaly, virtually segmenting the image to produce segmented image information.

Example 6 includes the subject matter of any one or more of the examples 1 to 5 and, optionally, wherein the steps further comprise:
displaying the segmented image information in offline and/or in real-time, during imaging of the tissue internal to the patient body.

Example 7 includes the subject matter of any one or more of the examples 1 to 6 and, optionally, wherein the image data is obtained using one or more of the following medical imaging modalities: ultrasound imaging, computer-tomography (CT) imaging, PET CT, Magnetic-Resonance Imaging (MRI), X-ray imaging or any combination of the aforesaid.

Example 8 includes the subject matter of example 7 and, optionally, wherein the image data is obtained using endoscopic imaging.

Example 9 includes the subject matter of any one or more of the examples 1 to 8 and, optionally, wherein the image data is descriptive of one or more organs, including: pancreas, kidneys, lung, bladder, prostate, esophagus, stomach, colon, skeleton, nervous system, any extraluminal (GI tract) structure such as lymph nodes, or any combination of the aforesaid.

Example 10 includes the subject matter of any one or more of the examples 1 to 9 and, optionally, wherein the analytics engine constitutes or includes a trained machine-learning (ML) model.

Example 11 includes the subject matter of example 10 and, optionally, wherein the trained ML model was trained with image data relating tissue anomalies of other mammalians.

Example 12 includes the subject matter of examples 10 and/or 11 and, optionally, wherein the trained ML model was trained to virtually segment image data, based on a plurality of training image datasets which were virtually segmented by at least two different annotators.

Example 13 includes the subject matter of example 12 and, optionally, wherein at least two the plurality of training image datasets is descriptive of a consensus Region-of-Interest (ROI) of a same image obtained from another mammalian.

Example 14 includes the subject matter of example 13 and, optionally, wherein a consensus ROI is an overlapping ROI of at least two ROIs delineated by at least two different annotators.

Example 15 includes the subject matter of any one or more of the examples 1 to 14 and, optionally, wherein the steps comprise: providing an all-lesion classification for a detected tissue anomaly.

Example 16 includes the subject matter of any one or more of the examples 1 to 15 and, optionally, wherein the steps comprise: distinguishing, for a detected anomaly, between a cancerous and non-cancerous anomaly.

Example 17 includes the subject matter of any one or more of the examples 1 to 16 and, optionally, wherein the steps comprise: for an anomaly identified as being non-cancerous, a probability to transform into a cancerous anomaly.

Example 18 includes the subject matter of any one or more of the examples 1 to 17 and, optionally, wherein the steps comprise determining a future time window within which the detected non-cancerous anomaly is expected to transform into a cancerous anomaly.

Example 19 includes the subject matter of any one or more of the examples 1 to 18 and, optionally, The computer program product of any one or more of the claims, wherein the steps comprise: recommending a diagnostic follow-up procedure for additionally assessing an identified tissue anomaly.

Example 20 includes the subject matter of any one or more of the examples 1 to 19 and, optionally, wherein the ML model is further trained based on one of the following data descriptive of information of the plurality of the other mammalians: medical, physiological data, socio-economic, behavioral patient characteristics, or any combination of the aforesaid.

Example 21 pertains to a method for analyzing tissue internal to a patient body, the method comprising:
receiving image data descriptive of tissue internal to a mammalian;
providing an image analysis engine with the received image data; and
determining for the received image data, by the image analysis engine,
whether imaged tissue includes a tissue anomaly or not.

Example 22 includes the subject matter of example 21 and, optionally, wherein the steps further comprise:
determining, for a detected anomaly, a type of anomaly.

Example 23 includes the subject matter of example 21 and/or 22 and, optionally, wherein the determining whether imaged tissue includes a tissue anomaly or not comprises determining a probability that imaged tissue comprises an anomaly.

Example 24 includes the subject matter of examples 21 to 23 and, optionally, wherein the determining whether imaged tissue includes a tissue anomaly or not comprises determining a probability that imaged tissue does not comprise an anomaly.

Example 25 includes the subject matter of examples 21 to 24 and, optionally, wherein the steps further comprise:
for a detected tissue anomaly, virtually segmenting the image to produce segmented image information.

Example 26 includes the subject matter of examples 21 to 25 and, optionally, wherein the steps further comprise: displaying the segmented image information in offline and/or in real-time, during imaging of the tissue internal to the patient body.

Example 27 includes the subject matter of examples 21 to 26 and, optionally, wherein the image data is obtained using one or more of the following medical imaging modalities: ultrasound imaging, computer-tomography (CT) imaging, PET CT, Magnetic-Resonance Imaging (MRI), X-ray imaging or any combination of the aforesaid.

Example 28 includes the subject matter of example 27 and, optionally, wherein the image data is obtained using endoscopic imaging.

Example 29 includes the subject matter of examples 21 to 28 and, optionally, wherein the image data is descriptive of one or more organs, including: pancreas, kidneys, lung, bladder, prostate, esophagus, stomach, colon, skeleton, nervous system, any extraluminal (GI tract) structure such as lymph nodes, or any combination of the aforesaid.

Example 30 includes the subject matter of examples 21 to 29 and, optionally, wherein the analytics engine constitutes or includes a trained machine-learning (ML) model.

Example 31 includes the subject matter of examples 21 to 30 and, optionally, wherein the trained ML model was trained with image data relating tissue anomalies of other mammalians.

Example 32 includes the subject matter of examples 21 to 31 and, optionally, wherein the trained ML model was trained to virtually segment image data, based on a plurality of training image datasets which were virtually segmented by at least two different annotators.

Example 33 includes the subject matter of examples 21 to 32 and, optionally, wherein each one of a plurality of training image datasets is descriptive of a consensus Region-of-Interest (ROI) of a same image obtained from another mammalian.

Example 34 includes the subject matter of examples 21 to 33 and, optionally, wherein a consensus ROI is an overlapping ROI of at least two ROIs delineated by at least two different annotators.

Example 35 includes the subject matter of examples 21 to 34 and, optionally, wherein the steps comprise:
providing an all-lesion classification for a detected tissue anomaly.

Example 36 includes the subject matter of examples 21 to 36 and, optionally, wherein the steps comprise: distinguishing, for a detected anomaly, between a cancerous and non-cancerous anomaly.

Example 37 includes the subject matter of examples 21 to 36 and, optionally, for an anomaly identified as being non-cancerous, a probability to transform into a cancerous anomaly.

Example 38 includes the subject matter of example 37 and, optionally, determining a future time window within which the detected non-cancerous anomaly is expected to transform into a cancerous anomaly.

Example 39 includes the subject matter of examples 21 to 38 and, optionally, wherein the steps comprise: recommending a diagnostic follow-up procedure for additionally assessing an identified tissue anomaly.

Example 40 includes the subject matter of examples 21 to 39 and, optionally, wherein the ML model is further trained based on one of the following data descriptive of information of the plurality of the other mammalians: medical, physiological data, socio-economic, behavioral patient characteristics, or any combination of the aforesaid.

It is noted that ML-based models discussed herein may be employed individually, or any combination, e.g., as an ensemble.

The methods described herein and illustrated in the accompanying diagrams shall not be construed in a limiting manner. For example, methods described herein may include additional or even fewer processes or operations in comparison to what is described herein and/or illustrated in the diagrams. In addition, method steps are not necessarily limited to the chronological order as illustrated and described herein.

Any digital computer system, apparatus, unit, device, module and/or engine exemplified herein can be configured or otherwise programmed to implement a method disclosed herein, and to the extent that the system, apparatus, module and/or engine is configured to implement such a method, it is within the scope and spirit of the disclosure. Once the system, apparatus, module and/or engine are programmed to perform particular functions pursuant to computer readable and executable instructions from program software that implements a method disclosed herein, it in effect becomes a special purpose computer particular to embodiments of the method disclosed herein. The methods and/or processes disclosed herein may be implemented as a computer program product that may be tangibly embodied in an information carrier including, for example, in a non-transitory tangible computer-readable and/or non-transitory tangible machine-readable storage device. The computer program product may be directly loadable into an internal memory of a digital computer, comprising software code portions for performing the methods and/or processes as disclosed herein.

The methods and/or processes disclosed herein may be implemented as a computer program that may be intangibly embodied by a computer readable signal medium. A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a non-transitory computer or machine-readable storage device and that can communicate, propagate, or transport a program for use by or in connection with apparatuses, systems, platforms, methods, operations and/or processes discussed herein.

The terms “non-transitory computer-readable storage device” and “non-transitory machine-readable storage device” encompasses distribution media, intermediate storage media, execution memory of a computer, and any other medium or device capable of storing for later reading by a computer program implementing embodiments of a method disclosed herein. A computer program product can be deployed to be executed on one computer or on multiple computers at one site or distributed across multiple sites and interconnected by one or more communication networks.

These computer readable and executable instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable and executable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable and executable instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The term “engine” may comprise one or more computer modules, wherein a module may be a self-contained hardware and/or software component that interfaces with a larger system. A module may comprise a machine or machines executable instructions. A module may be embodied by a circuit or a controller programmed to cause the systems, apparatuses and/or platforms to implement the method, process and/or operation as disclosed herein. For example, a module may be implemented as a hardware circuit comprising, e.g., custom VLSI circuits or gate arrays, an Application-Specific Integrated Circuit (ASIC), off-the-shelf semiconductors such as logic chips, transistors, and/or other discrete components. A module may also be implemented in programmable hardware devices such as field programmable gate arrays, programmable array logic, programmable logic devices and/or the like.

In the discussion, unless otherwise stated, adjectives such as “substantially” and “about” that modify a condition or relationship characteristic of a feature or features of an embodiment of the invention, are to be understood to mean that the condition or characteristic is defined to within tolerances that are acceptable for operation of the embodiment for an application for which it is intended.

Unless otherwise specified, the terms “substantially”, “‘about” and/or “close” with respect to a magnitude or a numerical value may imply to be within an inclusive range of −10% to +10% of the respective magnitude or value.

“Coupled with” can mean indirectly or directly “coupled with”.

It is important to note that the method may include is not limited to those diagrams or to the corresponding descriptions. For example, the method may include additional or even fewer processes or operations in comparison to what is described in the figures. In addition, embodiments of the method are not necessarily limited to the chronological order as illustrated and described herein.

Discussions herein utilizing terms such as, for example, “processing”, “computing”, “calculating”, “determining”, “establishing”, “analyzing”, “checking”, “estimating”, “deriving”, “selecting”, “inferring” or the like, may refer to operation(s) and/or process(es) of a computer, a computing platform, a computing system, or other electronic computing device, that manipulate and/or transform data represented as physical (e.g., electronic) quantities within the computer's registers and/or memories into other data similarly represented as physical quantities within the computer's registers and/or memories or other information storage medium that may store instructions to perform operations and/or processes. The term determining may, where applicable, also refer to “heuristically determining”.

It should be noted that where an embodiment refers to a condition of “above a threshold”, this should not be construed as excluding an embodiment referring to a condition of “equal or above a threshold”. Analogously, where an embodiment refers to a condition “below a threshold”, this should not be construed as excluding an embodiment referring to a condition “equal or below a threshold”. It is clear that should a condition be interpreted as being fulfilled if the value of a given parameter is above a threshold, then the same condition is considered as not being fulfilled if the value of the given parameter is equal or below the given threshold. Conversely, should a condition be interpreted as being fulfilled if the value of a given parameter is equal or above a threshold, then the same condition is considered as not being fulfilled if the value of the given parameter is below (and only below) the given threshold.

It should be understood that where the claims or specification refer to “a” or “an” element and/or feature, such reference is not to be construed as there being only one of that element. Hence, reference to “an element” or “at least one element” for instance may also encompass “one or more elements”.

Terms used in the singular shall also include the plural, except where expressly otherwise stated or where the context otherwise requires.

In the description and claims of the present application, each of the verbs, “comprise” “include” and “have”, and conjugates thereof, are used to indicate that the data portion or data portions of the verb are not necessarily a complete listing of components, elements or parts of the subject or subjects of the verb.

Unless otherwise stated, the use of the expression “and/or” between the last two members of a list of options for selection indicates that a selection of one or more of the listed options is appropriate and may be made. Further, the use of the expression “and/or” may be used interchangeably with the expressions “at least one of the following”, “any one of the following” or “one or more of the following”, followed by a listing of the various options.

As used herein, the phrase “A,B,C, or any combination of the aforesaid” should be interpreted as meaning all of the following: (i) A or B or C or any combination of A, B, and C, (ii) at least one of A, B, and C; (iii) A, and/or B and/or C, and (iv) A, B and/or C. Where appropriate, the phrase A, B and/or C can be interpreted as meaning A, B or C. The phrase A, B or C should be interpreted as meaning “selected from the group consisting of A, B and C”. This concept is illustrated for three elements (i.e., A, B, C), but extends to fewer and greater numbers of elements (e.g., A, B, C, D, etc.).

It is appreciated that certain features of the invention, which are, for clarity, described in the context of separate embodiments or examples, may also be provided in combination in a single embodiment. Conversely, various features of the invention, which are, for brevity, described in the context of a single embodiment, example and/or option, may also be provided separately or in any suitable sub-combination or as suitable in any other described embodiment, example or option of the invention. Certain features described in the context of various embodiments, examples and/or optional implementation are not to be considered essential features of those embodiments, unless the embodiment, example and/or optional implementation is inoperative without those elements.

It is noted that the terms “in some embodiments”, “according to some embodiments”, “for example”, “e.g.”, “for instance” and “optionally” may herein be used interchangeably.

The number of elements shown in the Figures should by no means be construed as limiting and is for illustrative purposes only.

It is noted that the terms “operable to” can encompass the meaning of the term “modified or configured to”. In other words, a machine “operable to” perform a task can in some embodiments, embrace a mere capability (e.g., “modified”) to perform the function and, in some other embodiments, a machine that is actually made (e.g., “configured”) to perform the function.

Throughout this application, various embodiments may be presented in and/or relate to a range format. It should be understood that the description in range format is merely for convenience and brevity and should not be construed as an inflexible limitation on the scope of the embodiments. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

While the invention has been described with respect to a limited number of embodiments, these should not be construed as limitations on the scope of the invention, but rather as exemplifications of some of the embodiments.

### References

1. Chari, S. T., et al., Early detection of sporadic pancreatic cancer: summative review. Pancreas, 2015. 44(5): p. 693-712.
2. Tanaka, M., et al., Revisions of international consensus Fukuoka guidelines for the management of IPMN of the pancreas. Pancreatology, 2017. 17(5): p. 738-753.
3. Abboud, Y., et al., Increasing Pancreatic Cancer Incidence in Young Women in the US: A Population-Based Time-Trend Analysis, 2001-2018. Gastroenterology, 2023.
4. Chiaravalli, M., M. Reni, and E. M. O'Reilly, Pancreatic ductal adenocarcinoma: State-of-the-art 2017 and new therapeutic strategies. Cancer Treat Rev, 2017. 60: p. 32-43.
5. Mizrahi, J. D., et al., Pancreatic cancer. Lancet, 2020. 395(10242): p. 2008-2020.
6. Lee, K. S., et al., Prevalence of incidental pancreatic cysts in the adult population on MR imaging. Am J Gastroenterol, 2010. 105(9): p. 2079-84.
7. Best, L. M., et al., Imaging modalities for characterising focal pancreatic lesions. Cochrane Database Syst Rev, 2017.4: p. CD010213.
8. Dhaliwal, A., et al., Efficacy of EUS-RFA in pancreatic tumors: Is it ready for prime time? A systematic review and meta-analysis. Endosc Int Open, 2020. 8(10): p. E1243-E1251.
9. Kitano, M., et al., Impact of endoscopic ultrasonography on diagnosis of pancreatic cancer. J Gastroenterol, 2019. 54(1): p. 19-32.
10. Yamamiya, A., et al., Interobserver Reliability of Endoscopic Ultrasonography: Literature Review. Diagnostics (Basel), 2020. 10(11).
11. Thomsen, M. M., et al., Accuracy and clinical outcomes of pancreatic EUS-guided fine-needle biopsy in a consecutive series of 852 specimens. Endosc Ultrasound, 2022. 11(4): p. 306-318.
12. King, D., et al., Rote of pancreatic cancer following a negative endoscopic ultrasound and associated factors. Endoscopy, 2022. 54(11): p. 1053-1061.
13. Hassan, C., et al., Performance of artificial intelligence in colonoscopy for adenoma and polyp detection: a systematic review and meta-analysis. Gastrointest Endosc, 2021. 93(1): p. 77-85 e6.
14. Kuwahara, T., et al., Usefulness of Deep Learning Analysis for the Diagnosis of Malignancy in Intraductal Papillary Mucinous Neoplasms of the Pancreas. Clin Transl Gastroenterol, 2019. 10(5): p. 1-8.
15. Zhang, J., et al., Deep learning-based pancreas segmentation and station recognition system in EUS: development and validation of a useful training tool (with video). Gastrointest Endosc, 2020. 92(4): p. 874-885 e3.
16. Tonozuka, R., et al., Deep learning analysis for the detection of pancreatic cancer on endosonographic images: a pilot study. J Hepatobiliary Pancreat Sci, 2020.
17. Voss, M., et al., Value of endoscopic ultrasound guided fine needle aspiration biopsy in the diagnosis of solid pancreatic masses. Gut, 2000. 46(2): p. 244-9.
18. Chen, S. C. and D. K. Rex, Endoscopist can be more powerful than age and male gender in predicting adenoma detection at colonoscopy. Am J Gastroenterol, 2007. 102(4): p. 856-61.
19. Yao, L., et al., Effect of artificial intelligence on novice-performed colonoscopy: a multicenter randomized controlled tandem study. Gastrointest Endosc, 2024. 99(1): p. 91-99 e9.
20. Das, A., et al., Digital image analysis of EUS images accurately differentiates pancreatic cancer from chronic pancreatitis and normal tissue. Gastrointest Endosc, 2008. 67(6): p. 861-7.
21. Kuwahara, T., et al., Artificial intelligence using deep learning analysis of endoscopic ultrasonography images for the differential diagnosis of pancreatic masses. Endoscopy, 2023. 55(2): p. 140-149.
22. Oh, S., et al., Automatic Pancreatic Cyst Lesion Segmentation on EUS Images Using a Deep-Learning Approach. Sensors (Basel), 2021. 22(1).
23. Iwasa, Y., et al., Automatic Segmentation of Pancreatic Tumors Using Deep Learning on a Video Image of Contrast-Enhanced Endoscopic Ultrasound. J Clin Med, 2021. 10(16).
24. Chu, L. C., M. G. Goggins, and E. K. Fishman, Diagnosis and Detection of Pancreatic Cancer. Cancer J, 2017. 23(6): p. 333-342.
25. Evans, D. B., B. A. Erickson, and P. Ritch, Borderline resectable pancreatic cancer: definitions and the importance of multimodality therapy. Ann Surg Oncol, 2010. 17(11): p. 2803-5.

1. A computer program product comprising program instructions for the execution of a method comprising the following steps, when the computer program is run on a computer:
receiving image data descriptive of tissue internal to a mammalian;
providing an image analysis engine with the received image data; and
determining for the received image data, by the image analysis engine,
whether imaged tissue includes a tissue anomaly or not.

2. (canceled)

3. (canceled)

4. (canceled)

5. (canceled)

6. (canceled)

7. (canceled)

8. (canceled)

9. (canceled)

10. The computer program product of claim 1, wherein the analytics engine constitutes or includes a trained machine-learning (ML) model.

11. (canceled)

12. The computer program product of claim 10, wherein the trained ML model was trained to virtually segment image data, based on a plurality of training image datasets which were virtually segmented by at least two different annotators.

13. The computer program product of claim 12, wherein each one of a plurality of training image datasets is descriptive of a consensus Region-of-Interest (ROI) of a same image obtained from another mammalian.

14. The computer program product of claim 13, wherein a consensus ROI is an overlapping ROI of at least two ROIs delineated by at least two different annotators.

15. (canceled)

16. The computer program product of claim 1, wherein the steps comprise:
distinguishing, for a detected anomaly, between a cancerous and non-cancerous anomaly.

17. The computer program product of claim 1, wherein the steps comprise:
for an anomaly identified as being non-cancerous, a probability to transform into a cancerous anomaly.

18. The computer program product of claim 17, wherein the steps comprise:
determining a future time window within which the detected non-cancerous anomaly is expected to transform into a cancerous anomaly.

19. The computer program product of claim 1, wherein the steps comprise:
recommending a diagnostic follow-up procedure for additionally assessing an identified tissue anomaly.

20. (canceled)

21. A method for analyzing tissue internal to a patient body, the method comprising
receiving image data descriptive of tissue internal to a mammalian;
providing an image analysis engine with the received image data; and
determining for the received image data, by the image analysis engine,
whether imaged tissue includes a tissue anomaly or not.

22. (canceled)

23. (canceled)

24. (canceled)

25. (canceled)

26. (canceled)

27. (canceled)

28. (canceled)

29. (canceled)

30. (canceled)

31. A system for analyzing tissue internal to a patient body, the system comprising:
a memory for storing executable instructions; and
a processor which, when executing executable instructions stored in the memory, results in the following steps:
receiving image data descriptive of tissue internal to a mammalian;
providing an image analysis engine with the received image data; and
determining for the received image data, by the image analysis engine,
whether imaged tissue includes a tissue anomaly or not.

32. (canceled)

33. (canceled)

34. (canceled)

35. (canceled)

36. (canceled)

37. (canceled)

38. (canceled)

39. (canceled)

40. The system of claim 31, wherein the analytics engine constitutes or includes a trained machine-learning (ML) model.

41. The system of claim 40, wherein the trained ML model was trained with image data relating tissue anomalies of other mammalians.

42. The system of claim 41, wherein the trained ML model was trained to virtually segment image data, based on a plurality of training image datasets which were virtually segmented by at least two different annotators.

43. The system of claim 42, wherein each one of a plurality of training image datasets is descriptive of a consensus Region-of-Interest (ROI) of a same image obtained from another mammalian.

44. The system of claim 43, wherein a consensus ROI is an overlapping ROI of at least two ROIs delineated by at least two different annotators.

45. (canceled)

46. The system of claim 31, wherein the steps comprise:
distinguishing, for a detected anomaly, between a cancerous and non-cancerous anomaly.

47. The system of claim 31,
wherein the steps comprise:
for an anomaly identified as being non-cancerous, a probability to transform into a cancerous anomaly.

48. The system of claim 47, wherein the steps comprise:
determining a future time window within which the detected non-cancerous anomaly is expected to transform into a cancerous anomaly.

49. The system of claim 31,
wherein the steps comprise:
recommending a diagnostic follow-up procedure for additionally assessing an identified tissue anomaly.

50. (canceled)
